(19) United States
(12) Patent Application Publication
(10) Pub. No.: US 2006/0264829 A1
(43) Pub. Date: Nov. 23, 2006

(54) DISPOSABLE FLUID CONTAINER WITH INTEGRATED PUMP MOTIVE ASSEMBLY

(75) Inventor: William M. DONALDSON, Newport News, VA (US)

Correspondence Address: NIXON & VANDERHYE, PC, 901 NORTH GLEBE ROAD, 11TH FLOOR, ARLINGTON, VA, 22203, US

(73) Assignee: PAR Technologies, LLC, Hampton, VA (US)

(21) Appl. No.: 11/382,502

(22) Filed: May 10, 2006

Related U.S. Application Data
(60) Provisional application No. 60/679,227, filed on May 10, 2005.
(60) Provisional application No. 60/700,736, filed on Jul. 20, 2005.

(51) Int. Cl. A61M 37/00; A61B 19/00; A61K 9/22

(52) U.S. Cl. 604/131; 604/890.1; 604/408

(57) ABSTRACT

A disposable fluid container (20) comprises an integrated pump motive assembly (50) for pumping fluid from an interior of the container. In some example embodiments, the integrated pump motive assembly (50) is situated at least partially within the container housing, e.g., at least partially or even completely submerged inside a fluid reservoir (30) defined by the container housing (22). In other example embodiments, the pump motive assembly is outside the fluid reservoir (30), yet still integrally formed with the container. In yet other embodiments, the pump motive assembly is in a lid (194) of the container. As one aspect of this disposable pump-integrated container technology, the pump motive assembly comprises a displaceable electrodynamic actuator, such as (for example) piezoelectric diaphragm pump, taking any of various configurations. As another and distinct aspect of this disposable pump-integrated container technology, the pump motive assembly is connected via an electrical lead (78) and/or terminal (80) to receive pump driving signals from outside the container.

This application claims the priority and benefit of U.S. Provisional Patent Application 60/679,227, filed May 10, 2005 and U.S. Provisional Patent Application 60/700,736, filed Jul. 20, 2005, and is related to simultaneously-filed U.S. patent application Ser. No. ______ (attorney docket: 4209-114), entitled “FLUID CONTAINER WITH INTEGRATED VALVE”, all of which are incorporated herein by reference in their entirety.

### BACKGROUND

1. Field of the Invention

The present invention pertains to the dispensing of fluids, particularly liquids, from a container which is inexpensive and disposable.

2. Related Art and Other Considerations

In myriad environments fluids are delivered or dispensed in controlled manner from disposable, inexpensive containers (e.g., bags, pouches, cartons, cartridges, just to name a few). The dispensing may be controlled to obtain a required or target dosage or amount over time, such as (for example) control of a medicament to a patient or an ingredient utilized in an industrial or other process.

Typically such control is achieved by connecting the disposable container to a host device, e.g., by various tubes or hoses, and allowing a pump at or near the host device or other device external to the container to draw fluid in metered manner from the container. When the container is closed and flexible, the pumping of the fluid essentially collapses the container. Such pump may be, for example, a peristaltic or other type of pump, and generally is rather sophisticated, bulky, and rather expensive. Over time successive containers of fluid are connected to the host device so that the external pump is utilized for the successive containers, typically having a working life comparable to that of the host device (e.g., on the order of years). In view of reuse of the host device, the pumps that are utilized are of the type that do not have direct contact with the fluid being dispensed or delivery. For example, a peristaltic pump has rollers or the like which contact a tube through which the fluid is supplied, but do not contact the fluid. In some fields and applications such as medicine and industrial processes, it is important (in view of reuse of the pump components) that the pump components not be contaminated by previous use, or in any way serve as a potential source of contamination or mixing for future jobs. In such host devices, the pumps that are utilized are never filled with fluids, but merely serve as indirect transmission agents for conveying fluid.

### BRIEF SUMMARY

A disposable fluid container comprises a fluid-contacting pump motive assembly which is integrated with the container for pumping fluid from an interior of the container to a host device. The disposable fluid container comprises a container body, and in some example embodiments an optional lid.

In some example embodiments, the integrated pump motive assembly is situated at least partially within the container body, e.g., at least partially or even completely submerged inside a fluid reservoir defined by the container body. In other example embodiments, the pump motive assembly is outside the fluid reservoir, yet still integrally formed with the container (e.g., on or between members such as films which form the container body). In being disposable, the container body is configured and/or used to be filled only once, and thereafter discarded.

In other example embodiments, the integrated pump motive assembly is situated at least partially within a lid of the container.

In the embodiments described herein, the pump motive assembly comprises at least a pump motive member such as one or more displaceable electrodynamic actuators, and may further include a pump housing with one or more inlet ports and one or more outlet ports, and/or inlet and/or outlet tubes connected to the convey fluid to/from the pump motive assembly. In some instances, the pump motive assembly may comprise essentially all elements of a pump except a pump drive circuit or comparable pump electronics. The pump motive assembly is fluid-handling in view of the fact that one or more elements of the pump motive assembly is/are essentially in direct contact with the fluid that is pumped.

The pump motive assembly preferably comprises a displaceable electrodynamic actuator. As used herein, a displaceable electrodynamic actuator can take the form of a piezoelectric member (such as a piezoelectric diaphragm); an actuator formed using electroreactive polymer(s) (EAP); an electrorestrictive pump; a shaped memory alloy; or, a magneto-restrictive element, to name a few examples. In essence, displaceable electrodynamic actuator encompasses any “smart” material which can used applied electrical energy to yield a mechanical displacement or deformation of itself, and (preferably) when subject to a mechanical force produces an electrical current.

The pump motive assembly can take various configurations. In some embodiments the pump motive assembly has a (preferably) rigid housing wherein the piezoelectric diaphragm is internally positioned for deflection to cause pumping in accordance with the drive signals applied thereto. Directionality of inlet and outlet flows relative to the pump housing varies accordingly to implementation. For example, in some implementations inlet and outlet ports of the pump housing are oriented so that inlet and outlet fluid flows are essentially axial relative to the displaceable electrodynamic actuator. In other implementations, the pump motive assembly can take the form of a bellows formed from two edge-bonded piezoelectric diaphragms.

As another and distinct aspect of this disposable pump-integrated container technology, the pump motive assembly is connected via an electrical lead and/or terminal to receive pump driving signals from outside the container. For example, the pump motive assembly can be connected to receive pump driving signals from the host device. Alternatively, the pump motive assembly can be connected to receive pump driving signals from a drive device which is distinct from the host device.

As another and distinct aspect of this disposable pump-integrated container technology, the container may include an identification or memory device for storing container information in electronic form. Contents of the identification or memory device (e.g., the container information stored in electronic form) can be accessed and utilized by a drive device or other external device via an electrical lead and/or terminal. The container information stored in electronic form can be one or more of container identification information, container fluid contents information, container volume information, lot number, serial number, origination (born on) date, expiration date, and the like. The identification or memory device can take the form of an EPROM or the like. The identification or memory device can be mounted to or within a pump housing. Alternatively, the identification or memory device can be mounted to another (e.g., non-pump) portion of the container (e.g., to members such as films which form the collapsible bladder).

In other example embodiments, the container comprises a container housing for at least partially enclosing the container body. In some implementations of this embodiment, the container housing is substantially rigid and has a bleed valve to permit collapsing of the bladder. The container body can take the form of a collapsible bladder.

### BRIEF DESCRIPTION OF THE DRAWINGS

The foregoing and other objects, features, and advantages of the invention will be apparent from the following more particular description of preferred embodiments as illustrated in the accompanying drawings in which reference characters refer to the same parts throughout the various views. The drawings are not necessarily to scale, emphasis instead being placed upon illustrating the principles of the invention.

FIG. 1A is a front view of a disposable fluid container according to a first example embodiment.

FIG. 1B is a right sectioned view of the disposable fluid container of FIG. 1A taken along line 1B-1B.

FIG. 1C is a top sectioned view of the disposable fluid container of FIG. 1A taken along line 1C-1C.

FIG. 2A is an isometric view of an example piezoelectric pump motive assembly according to an example embodiment.

FIG. 2B is a side sectioned view of the example piezoelectric pump motive assembly of FIG. 2A.

FIG. 3A, FIG. 3B, and FIG. 3C are front views showing differing embodiments of fluid handling systems comprising both a disposable fluid container and a host or device.

FIG. 4 is a front view of a disposable fluid container according to another example embodiment.

FIG. 5A is a front view of a disposable fluid container according to another example embodiment.

FIG. 5B is a partial, sectioned right side view of the disposable fluid container of FIG. 5A.

FIG. 6A is a front view of a disposable fluid container according to another example embodiment.

FIG. 6B is a bottom sectioned view of the disposable fluid container of FIG. 6A taken along line 6B-6B.

FIG. 7A is an isometric view of an example piezoelectric pump motive assembly according to another example embodiment.

FIG. 7B is a side sectioned view of the example piezoelectric pump motive assembly of FIG. 7A.

FIG. 8A is an isometric view of an example disposable fluid container according to another example embodiment.

FIG. 8B is a side sectioned view of the example disposable fluid container of FIG. 8A taken along line 8B-8B.

FIG. 9A is a cross sectioned side view of a pump motive assembly according to another example embodiment.

FIG. 9B is a sectioned top view of the pump motive assembly of FIG. 9A taken along line 9B-9B.

FIG. 9C is a section front view of the pump motive assembly of FIG. 9A taken along line 9C-9C.

FIG. 10A is a front view of a disposable fluid container according to another example embodiment.

FIG. 10B is an enlarged partial right sectioned view of the disposable fluid container of FIG. 10A taken along line 10B-10B.

FIG. 10C is a bottom sectioned view of the disposable fluid container of FIG. 10A taken along line 10C-10C.

FIG. 11 is a front view of a disposable fluid container according to another example embodiment.

FIG. 12 is a front view of a disposable fluid container according to another example embodiment.

FIG. 13 is a front view of a disposable fluid container according to another example embodiment.

FIG. 14 is a front sectional view of a disposable fluid container according to another example embodiment.

FIG. 15 is a front sectional view of a disposable fluid container according to another example embodiment.

FIG. 16 is a front sectional view of a disposable fluid container according to another example embodiment.

FIG. 17 is a front sectional view of a disposable fluid container according to another example embodiment.

FIG. 18 is a front sectional view of a disposable fluid container according to another example embodiment.

FIG. 19A and FIG. 19B are top and bottom isometric views, respectively, of a pump motive assembly according to another embodiment.

FIG. 20 is an exploded isometric view of the pump motive assembly of FIG. 19A and FIG. 19B.

### DETAILED DESCRIPTION OF THE DRAWINGS

In the following description, for purposes of explanation and not limitation, specific details are set forth such as particular architectures, interfaces, techniques, etc. in order to provide a thorough understanding of the present invention. However, it will be apparent to those skilled in the art that the present invention may be practiced in other embodiments that depart from these specific details. In other instances, detailed descriptions of well-known devices, circuits, and methods are omitted so as not to obscure the description of the present invention with unnecessary detail. Moreover, individual function blocks for drive circuits are shown in some of the figures. Those skilled in the art will appreciate that the functions of drive circuits or the like may be implemented using individual hardware circuits, using software functioning in conjunction with a suitably programmed digital microprocessor or general purpose computer, using an application specific integrated circuit (ASIC), and/or using one or more digital signal processors (DSPs). As numerous, example, illustrative, non-limiting embodiments are described, it should be appreciated that comparable or analogous elements utilized in one or more embodiments bear the same or a similar reference numeral, in some cases the reference numeral being suffixed with a parenthetical indication of a figure number corresponding to the embodiment. Moreover, features or aspects of an example implementation or embodiment may be incorporated into another embodiment, although such incorporation may not be mentioned at time of description of either embodiment per se.

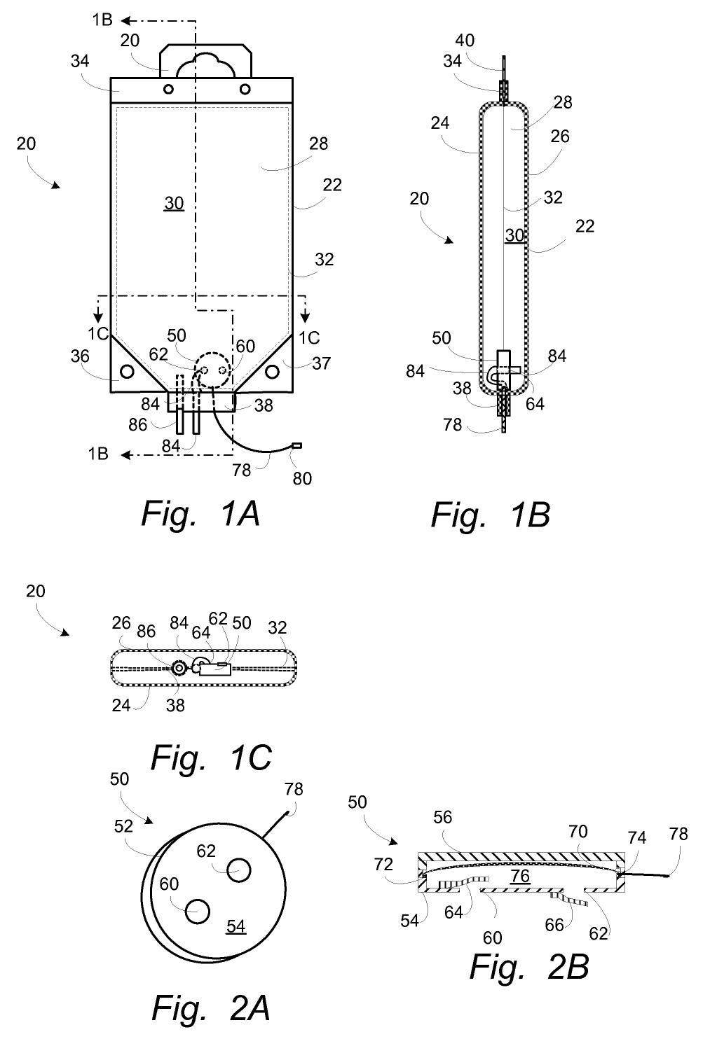

FIG. 1A, FIG. 1B, and FIG. 1C show a first example embodiment of a disposable fluid container 20. The disposable fluid container 20 has a container body 22 which, in the example illustrated embodiments hereof, is a multi-ply or multi-layer assembly comprised of flexible plys or layers (e.g., front layer 24 and rear layer 26, both shown in FIG. 1B and FIG. 1C). Given the flexible nature of the ply or layers forming the multi-ply or multi-layer assembly, in at least some implementations at least a portion of the container body 22 forms or defines a collapsible bladder 28. The collapsible bladder 28 defines a fluid reservoir 30 which is mainly bounded by a seam 32 which joins front layer 24 and rear layer 26.

The shape and configuration of the container body 22, collapsible bladder 28, and fluid reservoir 30 defined therein (e.g., between the layers 24, 26) can vary depending on implementation, only an example being shown in the embodiment of FIG. 1A and other embodiments. The container body 22 can optionally comprise or have attached thereto additional features or accessories, such as one or more flanges 34, 36, 37, and 38, and a handle or hanger 40. In the illustrated implementation of the first embodiment, the flanges include top flange 34; left bottom corner flange 36; right bottom corner flange 37; and, bottom central flange 38. Preferably the flanges 34, 36, 37, and 38 are formed by union or bonding of front layer 24 and rear layer 26. Unlike the region of collapsible bladder 28, which expands to accommodate fluid, the flanges 34, 36, 37, and 38 are relatively flat regions.

The container body 22 can be formed from any suitable material, examples of which are provided subsequently. In some example implementations, one or both of front layer 24 and rear layer 26 can be transparent, thereby affording visibility of the fluid contained in fluid reservoir 30 and other internal contents and/or features of disposable fluid container 20. Even though the internal contents and/or features may be visible because of such transparency, in the drawings the internal contents and/or features are illustrated with broken lines to reflect their internal location.

In addition to its collapsible bladder 28, disposable fluid container 20 comprises a pump motive assembly 50 which is integrated with the container body 22. The integrated pump motive assembly 50 serves for pumping fluid from an interior of container body 22, e.g., from an interior of the bladder, i.e., from fluid reservoir 30, to a host device. In the embodiments described herein, pump motive assembly comprises at least a pump motive member such as one or more displaceable electrodynamic actuators, and may further include a pump housing with one or more inlet ports and one or more outlet ports, and/or inlet and/or outlet tubes connected to the convey fluid to/from the pump motive assembly. In some instances, the pump motive assembly may comprise essentially all elements of a pump except a pump drive circuit or comparable pump electronics. The pump motive assembly is fluid-handling in view of the fact that one or more elements of the pump motive assembly is/are essentially in direct contact with the fluid that is pumped. For example, a displaceable electrodynamic actuator of the pump motive assembly acts directly upon the fluid. If the displaceable electrodynamic actuator were to be covered and separated from the fluid by a protective layer or intermediate layer or the like, as used herein the pump motive assembly is still integrated with the container body as long as the protective layer or intermediate layer or comparable intermediate component itself comprises the pump motive assembly and/or is integrated with the container body.

Thus, the pump motive assemblies of the embodiments described herein and/or otherwise encompassed hereby are integrated with the disposable fluid container. As mentioned above, the pump motive assembly preferably comprises a displaceable electrodynamic actuator. As used herein, a displaceable electrodynamic actuator can take the form of a piezoelectric member (such as a piezoelectric diaphragm); an actuator formed using electroreactive polymer(s) (EAP); an electrorestrictive pump; a shaped memory alloy; or, a magneto-restrictive element, to name a few examples. In essence, displaceable electrodynamic actuator encompasses any “smart” material which can used applied electrical energy to yield a mechanical displacement or deformation of itself, and (preferably) when subject to a mechanical force produces an electrical current.

In the example embodiment of FIG. 1A, pump motive assembly 50 is partially or completely submerged inside fluid reservoir 30 defined by collapsible bladder 28. In particular, pump motive assembly 50 is situated at a bottom central region of fluid reservoir 30, between left bottom corner flange 36 and right bottom corner flange 37 and above bottom central flange 38. In variations of this example embodiment, it should be understood that the integrated pump motive assembly can instead be situated only partially within collapsible bladder 28, e.g., a portion of pump motive assembly 50 may be encased between front layer 24 and rear layer 26 in a seam or flange region, for example.

In one example, non-limiting embodiment described herein, the integrated pump motive assembly pump comprises a piezoelectric diaphragm. The piezoelectric diaphragm is just one example of a suitable displaceable electrodynamic actuator for use with the pump motive assembly. Even within the piezoelectric diaphragm subclass of displaceable electrodynamic actuators, the piezoelectric pump motive assemblies can take various configurations. In the particular example embodiment shown in FIG. 1A-FIG. 1C a pump motive assembly 50 such as that shown in FIG. 2A and FIG. 2B can be utilized. The pump motive assembly 50 comprises a pump housing 52, which is preferably but not necessarily rigid. The pump housing 52 has an essentially disk or cylindrical shape. In one example implementation, pump housing 52 has a diameter of about 28 mm [1.125 inch] in diameter or less and an axial dimension of about 6 mm [0.25 inch] or less. The pump housing 52 comprises a pump bottom wall 54 and a pump top wall 56. The pump bottom wall 54 has both an inlet port 60 and an outlet port 62 formed therein.

As shown in FIG. 2B, an inlet valve 64 may be positioned for selective opening and closing of inlet port 60, an outlet valve 66 may be positioned for selective opening and closing of outlet port 62. Typically only one of the inlet port 60 and outlet port 62 need have a valve. In some implementations, particularly those in which the pump motive assembly is submerged or immersed in the fluid reservoir, the outlet port 62 port will generally have the valve. To avoid flow-through of the pump motive assembly in such immersed, one-valve environments, discharge from the pump motive assembly can be prevented by clamping or otherwise closing an outlet tube or the like downstream from the pump motive assembly.

A piezoelectric diaphragm member 70 is circumferentially retained in pump housing 52 between pump housing 52 and pump bottom wall 54, e.g., by spacer 72 and O-ring or spacer 74. On its underside piezoelectric diaphragm member 70 defines a pumping chamber 76. Thus, outlet port 62 and inlet valve 64 of pump housing 52 are oriented so that inlet and outlet fluid flows are essentially axial relative to the piezoelectric diaphragm 70.

When a piezoelectric diaphragm is used as the displaceable electrodynamic actuator, preferably the piezoelectric diaphragm member 70 is a ruggedized laminated piezoelectric member comprising a piezoelectric wafer core. The piezoelectric wafer core has a metallic layer laminated to at least one and possibly both of its major surfaces. Preferably a lower surface of the piezoelectric diaphragm member 70 which faces the pumping chamber 76 has a stainless steel layer bonded thereto, while an upper surface of piezoelectric diaphragm member 70 has a fluid non-reactive surface bonded thereto (e.g., an aluminum layer bonded thereto for some example applications).

Two electrodes of the piezoelectric diaphragm member 70 are connected by two electrical leads, collectively shown as electrical lead 78, for receipt of driving signals. The electrical lead 78 may terminate in a connector or electrical terminal 80 (see FIG. 1A), if desired. As used herein, “electrical terminal” is understood to encompass other forms or devices for electrical interconnection, such as (by way of non-limiting example) a pigtail, pogo pins, or spring-biased electrodes.

Further details of pump motive assembly 50, including structure and assembly of the pump housing 52 and of its piezoelectric diaphragm member 70, as well as other variations and implementations, are understood with reference to one or more of the following (all of which are incorporated herein by reference in their entirety): PCT Patent Application PCT/US01/28947, filed 14 Sep. 2001; U.S. patent application Ser. No. 10/380,547, filed Mar. 17, 2003, entitled “Piezoelectric Actuator and Pump Using Same”; U.S. patent application Ser. No. 10/380,589, filed Mar. 17, 2003, entitled “Piezoelectric Actuator and Pump Using Same”; U.S. patent application Ser. No. 11/270,647 filed Apr. 13, 2006 by Tietze et al., entitled “PIEZOELECTRIC DIAPHRAGM ASSEMBLY WITH CONDUCTORS ON FLEXIBLE FILM”.

As mentioned above, in the embodiment of FIG. 1A-FIG. 1C the pump motive assembly 50 is situated in a bottom central region of fluid reservoir 30. Preferably the entire pump motive assembly 50 is immersed in fluid reservoir 30, leaving inlet port 60 exposed (e.g., immersed) and with its inlet valve 64 operative for admitting fluid into pumping chamber 76. The outlet port 62 of pump motive assembly 50 is connected to outlet tube 84. The outlet tube 84 travels around the back and downward from outlet port 62 and through bottom central flange 38, between front layer 24 and rear layer 26, and extends from and beyond bottom central flange 38 (see FIG. 1A).

The bottom central flange 38 thus serves as a clamp or retainer for outlet tube 84, without restricting flow through outlet tube 84. If desired, a flow restrictor, valve, or shut-off can be provided on outlet tube 84 below bottom central flange 38. In addition, bottom central flange 38 serves as a clamp or retainer for electrical lead 78. The electrical lead 78 extends through pump housing 52 from a periphery of piezoelectric diaphragm member 70 downward and through bottom central flange 38 (e.g., between front layer 24 and rear layer 26), eventually terminating in electrical connector 80. Further, if desired, an additional (optional) tube, such as fill tube 86, can be retained or clamped by bottom central flange 38. A first end of fill tube 86 protrudes into fluid reservoir 30; a second end of fill tube 86 extends beyond bottom central flange 38.

As another and distinct aspect of this disposable pump-integrated container technology, pump motive assembly 50 is connected via electrical lead 78 and terminal 80 to receive pump driving signals from outside disposable fluid container 20. For example, as explained with reference to several non-limiting examples provided below, pump motive assembly 50 can be connected to receive pump driving signals from a host device or utility device.

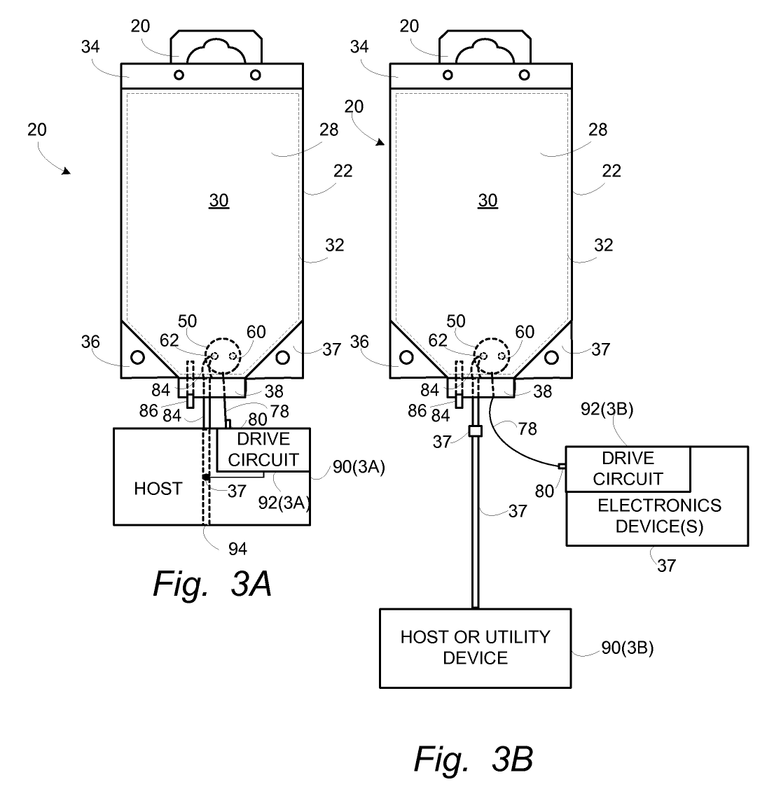

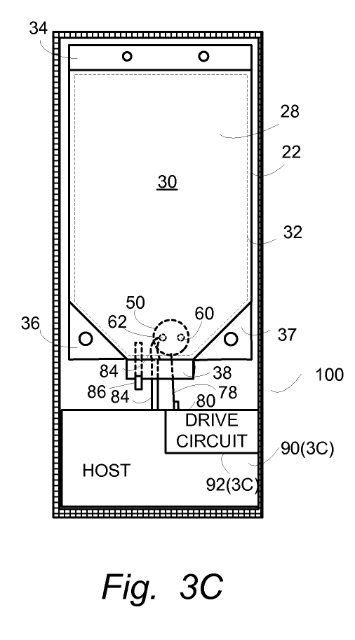

FIG. 3A shows disposable fluid container 20 connected to host device 90(3A). In the FIG. 3A embodiment, host device 90(3A) is of a type that receives fluid via outlet tube 84 from disposable fluid container 20 and transmits the received fluid through host internal channel 94 for discharge to another device, e.g., a utilization device. The host device 90(3A) includes a drive circuit 92(3A) which supplies driving signals to pump motive assembly 50 of disposable fluid container 20 over electrical lead 78. If desired, a flowmeter or other type of sensor 96 can be positioned in host internal channel 94 and be electrically connected to drive circuit 92(3A). Such sensor 96 can be utilized by the drive circuit 92(3A) to govern application of pumping signals to the pump motive assembly.

FIG. 3B shows disposable fluid container 20 connected to host or utilization device 90(3B). In the FIG. 3B embodiment, host or utilization device 90(3B) is more remote from disposable fluid container 20. To cater for the more remote location, outlet tube 84 of disposable fluid container 20 is connected by a fluidic coupler 98 to extension tube 99. A drive device such as drive circuit 92(3B) is situated distinct from host or utility device 90(3B), e.g., in a separate electronics cabinet or the like.

FIG. 3C shows a variation of the disposable fluid container of FIG. 1A situated in a host frame or bed 100 of a host device. The host frame 100 essentially encompasses the disposable fluid container as well as the utility device 90(3C). The host frame 100 can have an unillustrated cover, as well as other internal and external features.

Non-limiting examples of driving signals and drive circuits for any embodiment described herein and other embodiments are described in U.S. patent application Ser. No. 10/815,978, filed Apr. 2, 2004 by Vogeley et al., entitled “Piezoelectric Devices and Methods and Circuits for Driving Same”, which is incorporated herein by reference in its entirety, or by documents referenced and/or incorporated by reference therein.

As another and distinct aspect of this disposable pump-integrated container technology, usable with any or all of the embodiments described herein and other embodiments envisioned hereby, the container may include an identification or memory device for storing container information in electronic form. Contents of the identification or memory device (e.g., the container information stored in electronic form) can be accessed and utilized by a drive device or other external device via an electrical lead and/or terminal.

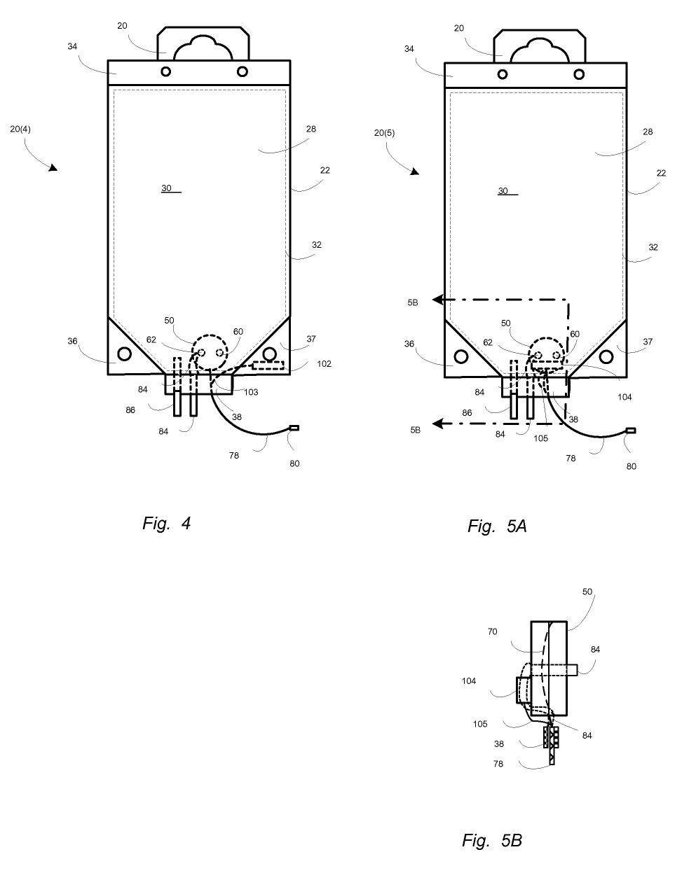

FIG. 4 shows a disposable fluid container 20(4) which essentially resembles disposable fluid container 20 of FIG. 1A, but which additionally has an identification or memory device in the form of EPROM 102. The EPROM 102 can be mounted to non-pump portions of the disposable fluid container 20, e.g., to members such as films which form the collapsible bladder, e.g., front layer 24 and rear layer 26. In the particular example shown in FIG. 4, EPROM 102 is mounted to and carried on right bottom corner flange 37. The EPROM 102 is connected to a lead 103 which travels to join the lead from the pump motive assembly 50 and is included in electrical lead or cable 78. Other mounting positions for EPROM 102 are also possible, such as left bottom corner flange 36 or bottom central flange 38, for example.

FIG. 5A and FIG. 5B show another embodiment of a disposable fluid container which includes an identification or memory device, particularly disposable fluid container 20(5). In 20(5) an EPROM 104 is carried on pump motive assembly 50, e.g., on an exterior of pump housing 52 such as on pump top wall 56. The EPROM 104 is connected to a lead 105 which travels to join the lead from the pump motive assembly 50 and is included in electrical lead or cable 78. Alternatively, the EPROM 104 could be carried within pump housing 52, e.g., on an interior surface of pump top wall 56, for example.

For embodiments with identification or memory devices, the container information stored in electronic form in the identification or memory device can be one or more of container identification information (e.g., one or more of type of container, model number, manufacturer identifier, serial number), container fluid contents information (e.g., one or more of identifier or descriptor of chemical composition of fluid, brand name of fluid), container volume information (volume at full, remaining volume or volume dispensed, ideal dispense ratio), lot number, serial number, origination (born on) date, expiration date, and the like.

One potential purpose of the container information stored by the identification or memory device is to assure that an appropriate disposable fluid container has been connected to the host or utilization device. The container information can thus prevent unauthorized or inappropriate containers from being actively utilized with the host device, or (in some environments) to prevent reuse of already-utilized containers. Accordingly, it should be appreciated that the identification or memory device need not be limited to an EPROM or comparable semiconductor memory. Any suitable device which provides an identification of the disposable fluid container can be utilized, such as (for example) a simple circuit that provides a predetermined resistive or other electrical value. Identification pins on a connector can be electrically strapped to either power or ground supply rails via zero ohm resistors to signify a binary code. In such case, “n” number of pins will allow for 2ⁿ number of combinations. These combinations can be detailed in a look up table.

In some example embodiments, the host or utility device can do more than access contents of the information or memory device carried by the disposable fluid container. For example, the host or utility device can write or record data in the information or memory device, e.g., in an EPROM. For example, the host or utility device can calculate or otherwise discern how much of the fluid has been pumped from (or, alternatively, remains in) fluid reservoir 30 and record or write such amount in the memory device (EPROM). Such indication of used or remaining amount is particularly helpful in embodiments in which the disposable fluid container is permitted to be re-used after initial or previous only partial usage, especially with other host or utility devices that otherwise would not know the history or amount used/remaining in the disposable fluid container. In such situations, the other or second host or utility device can ascertain from the memory device (carried on-board the disposable fluid container) how much fluid has been used (or, alternatively, remains) after previous usage by another host device. Computations or calculations directed to the amount used/remaining can be performed by appropriate electronics in the host or utility device by taking into account, e.g., a number of strokes of the displaceable electrodynamic actuator, a signal value utilized to drive the stroke(s), and a corresponding displacement or fluid discharge amount associated with the signal value. The host or utility device can include a display or other output device which indicates, e.g., graphically, the amount of fluid used or remaining in the disposable fluid container, in similar manner as do conventional printers regarding an amount of ink remaining in an ink cartridge.

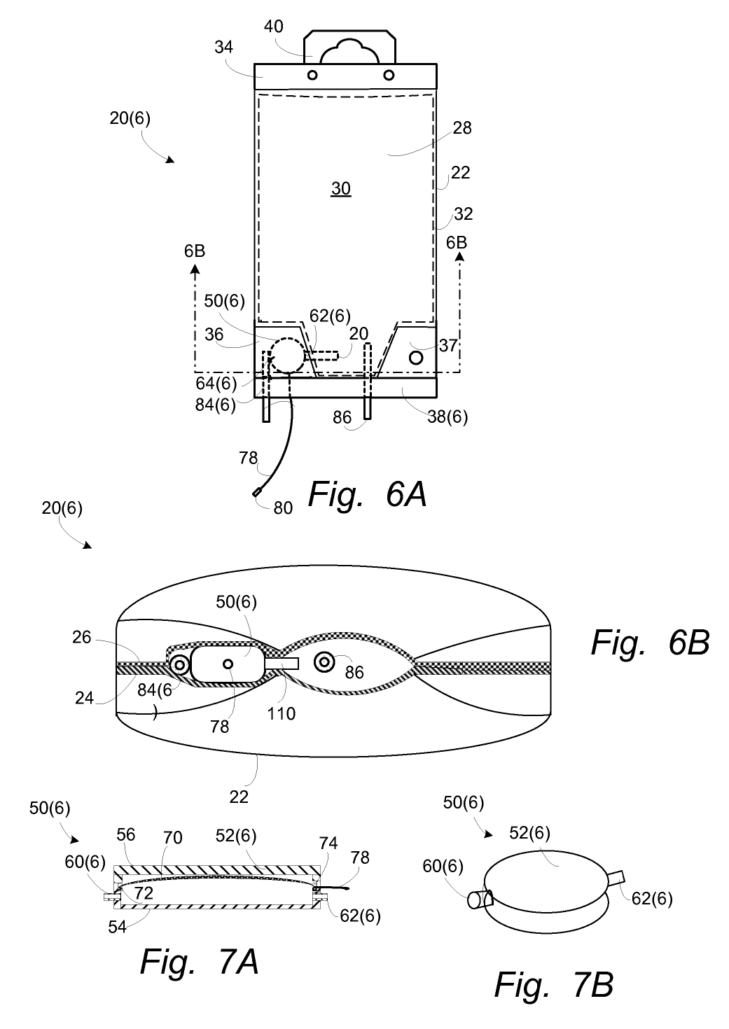

FIG. 6A and FIG. 6B illustrate another example embodiment of a disposable fluid container, e.g., disposable fluid container 20(6). In the disposable fluid container disposable fluid container 20(6), pump motive assembly 50(6) is outside the fluid reservoir 30, yet still integrally formed with the container 20(6), e.g., on or between members such as films which form the container body 22 with its collapsible bladder such as front layer 24 and rear layer 26. In the embodiment of FIG. 6A and FIG. 6B, the pump motive assembly 50(6) is formed in the region of left bottom corner flange 36, and is sandwiched between front layer 24 and rear layer 26 as shown in FIG. 6B.

FIG. 6A and FIG. 6B also illustrate that pump motive assembly 50(6) can alternatively take another configuration than that of pump motive assembly 50 of FIG. 1A. As shown in more detail in FIG. 7A and FIG. 7B, pump motive assembly 50(6) differs from pump motive assembly 50 by directionality of inlet and outlet flows relative to the pump housing. For pump housing 52(6) of FIG. 7A and FIG. 7B, the inlet port 60(6) and outlet port 62(6) are positioned and configured so that fluid flow through inlet port 60(6) and outlet port 62(6) is essentially in a radial direction of the piezoelectric diaphragm member 70 of pump motive assembly 50(6). As understood from a previous discussion, the inlet port 60(6) and outlet port 62(6) can have internal valves therein.

Since pump motive assembly 50(6) is situated on a flange such as left bottom corner flange 36 and is outside of fluid reservoir 30, outlet port 62(6) has an inlet tube or inlet port extension 110 provided thereon. The inlet port extension 110 extends through a sealed aperture of front layer 24 of container body 22 so that a distal end of inlet port extension 110 protrudes into fluid reservoir 30. Accordingly, fluid in fluid reservoir 30 can be drawn via inlet port extension 110 into pump motive assembly 50(6).

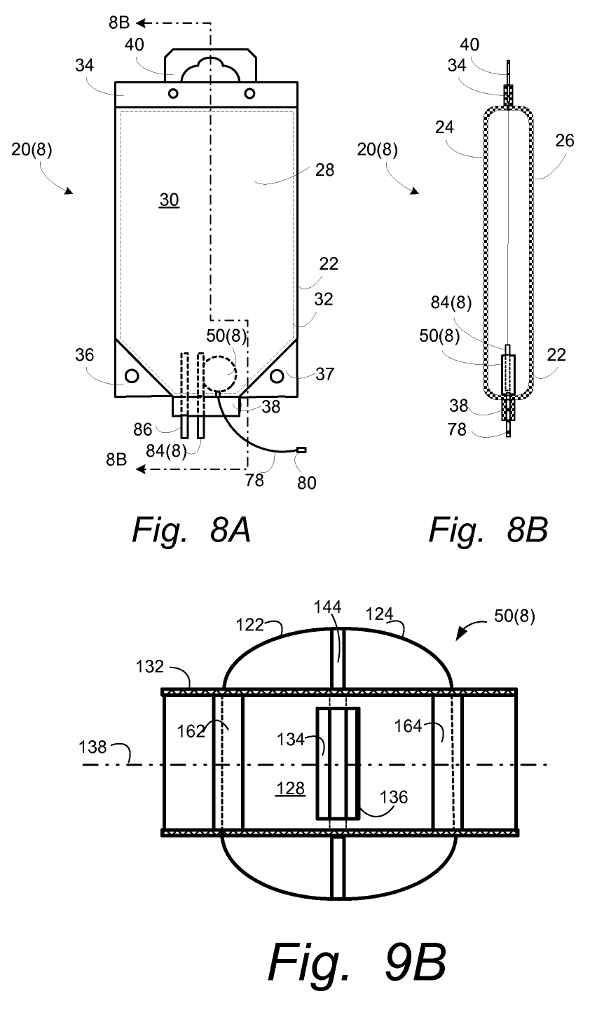

In other implementations, the pump motive assembly can take the form of a bellows formed from two edge-bonded piezoelectric diaphragms. For example, FIG. 8A and FIG. 8B illustrate another example embodiment of a disposable fluid container, particularly disposable fluid container 20(8), which comprises pump motive assembly 50(8) which is located within fluid reservoir 30.

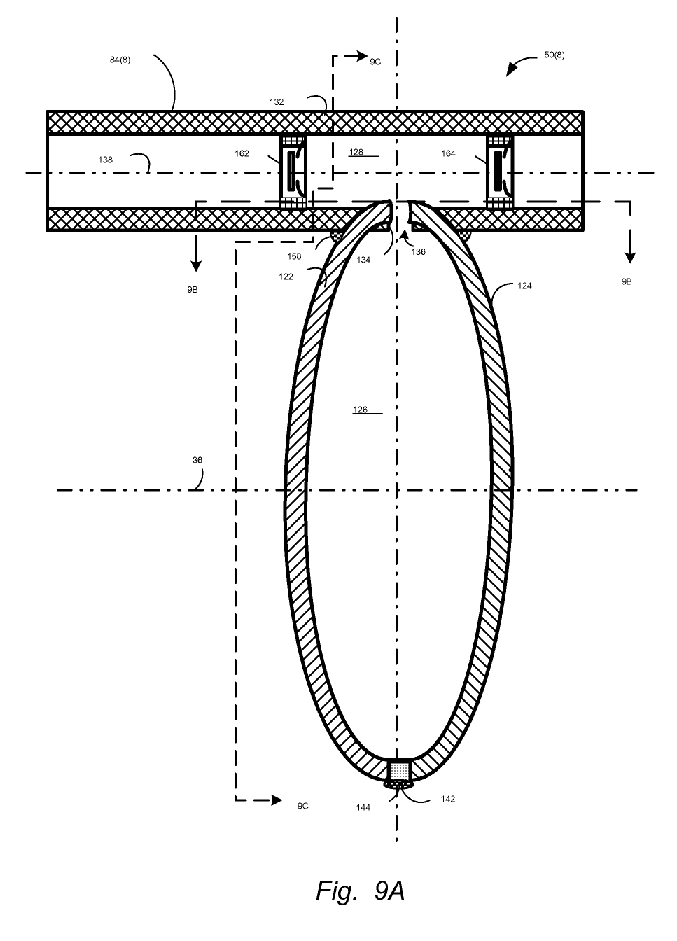

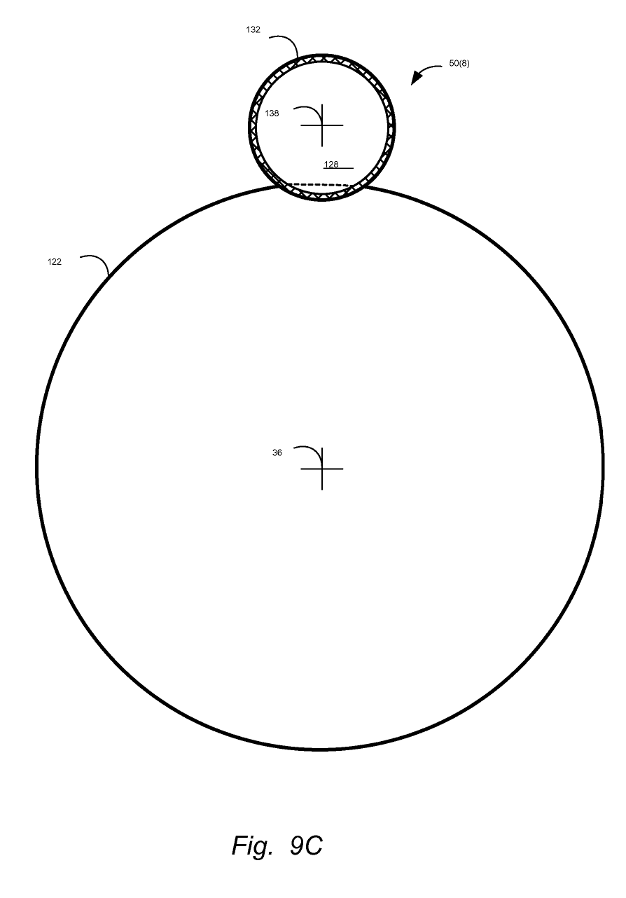

One example configuration of pump motive assembly 50(8) with its two edge-bonded piezoelectric diaphragms is illustrated in FIG. 9A-FIG. 9C. Such pump motive assembly is described below, and in more detail in U.S. patent application Ser. No. 11/024,943, filed Dec. 30, 2004 by Vogeley et al., entitled “PUMPS WITH DIAPHRAGMS BONDED AS BELLOWS”, which is incorporated herein by reference in its entirety, or by documents referenced and/or incorporated by reference therein. The pump motive assembly 50(8) has diaphragm 122 and diaphragm 124 bonded to form bellows chamber 126. The bellows chamber 126 is auxiliary to and communicates with a separate pumping chamber 128. The pumping chamber 128 is defined by a chamber housing 132 positioned above the diaphragm assembly. In the illustrated implementation, chamber housing 132 takes the form of a cylindrical tube which has a central axis 138. Other shapes and configurations for chamber housing 132 are possible.

In one example mode of fabrication, communication between bellows chamber 126 and pumping chamber 128 is facilitated by an aperture or slit 134 formed or provided in and through the bottom wall of chamber housing 132, as well as an opening or aperture 136 formed in the bonding interface of first diaphragm 122 and second diaphragm 124. In one illustrated implementation with a cylindrical chamber housing 132, the slit 134 is provided transverse to central axis 138 and at the bottom of the chamber housing 132 (see FIG. 9A, FIG. 9B, and FIG. 9C). The communication aperture 136 of the diaphragm assembly may be realized by omitting or removing the sealing gasket 142 and epoxy 144 at the crown (or other appropriate location) along the edge 148 and edge 150 of the diaphragm 122 and diaphragm 124, respectively. The length of the omission or removal of the sealing gasket 142 and epoxy 144 is on the order of the length of the slit 134 provided on the underside of chamber housing 132. Preferably a region of the upper portion of the diaphragm assembly which bears the communication aperture 136 is inserted into housing slit 134 so that a small tangential portion of edge 148 of first diaphragm 122 and a small tangential portion of edge 150 of second diaphragm 124 protrude into or are flush with edges of housing slit 134, as shown in FIG. 9B, thereby permitting communication, e.g., a pressure waveform through communication aperture 136 between bellows chamber 126 and pumping chamber 128. The diaphragm assembly can be secured to chamber housing 132 and the interface between communication aperture 136 and slit 134 sealed, if necessary, by an appropriate bonding or sealing medium 158, such as epoxy, for example.

Other modes of providing communication between bellows chamber 26 and pumping chamber 128 are also encompassed hereby. For example, a separate tube, passageway, or the like may connect bellows chamber 26 and pumping chamber 128, and may be positioned at locations other than at the bond of diaphragm 22 and diaphragm 24.

The pumping chamber 128 of pump motive assembly 20(8) has both an inlet valve 162 and an outlet valve 164. For some implementations and applications the inlet valve 162 and outlet valve 164 may preferably be positioned in pumping chamber 128 close together in order to facilitate priming of pump motive assembly 50(8) and performance. In other systems that do not require priming, it may instead be beneficial to position the inlet and outlet valves away from the bellows, e.g., one or more valves somewhere in a cooling loop with one or more bellows at different points in the loop.

In pump motive assembly 50(8), action of the diaphragm assembly, e.g., first diaphragm 122 and second diaphragm 124, generates a pressure waveform in the bellows chamber 126. The pressure waveform is communicated through aperture 136 and slit 134 and into pumping chamber 128 for selectively causing impulsion of the fluid through inlet valve 162 into the pumping chamber 128, and expulsion of the fluid through outlet valve 164 from the pumping chamber 128.

The chamber housing 132 of pump motive assembly 50(8) with its pumping chamber 128 can be stationary, with the first diaphragm 122 and the second diaphragm 124 being suspended from the pumping chamber and externally essentially unconstrained. Thus, both first diaphragm 122 and second diaphragm 124 are active.

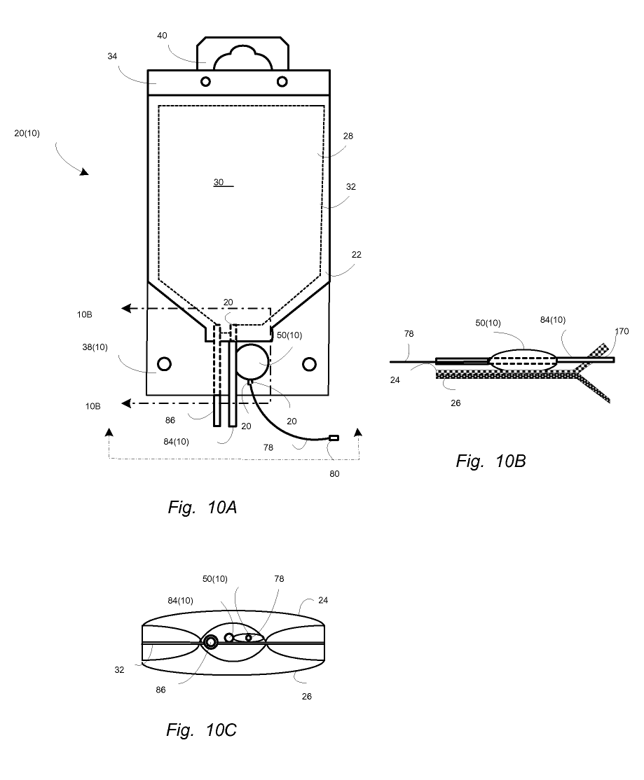

As shown in FIG. 8A and FIG. 8B, an extended segment of chamber housing 132 serves as both the outlet tube 84(8) and the inlet tube for the bellows embodiment. In particular, a first end of chamber housing 132 serves as outlet tube 84(8) by extending through the bottom central flange 38. A second end of chamber housing 132, which extends upwardly from the pump body into fluid reservoir 30, serves as the inlet tube to the pump motive assembly 50(8). Since there are two diaphragms in the pump motive assembly 50(8), two sets of two electrical leads are included in main electrical lead 78.

Whereas in the embodiment of FIG. 8A and FIG. 8B the bellows-type pump is situated in fluid reservoir 30, FIG. 10A-FIG. 10C illustrate an example embodiment wherein a bellows-type pump motive assembly 50(10) is integrated with disposable fluid container 20(10) and positioned outside of fluid reservoir 30. In the example embodiment of FIG. 10A-FIG. 10C, the pump motive assembly 50(10) is situated on a bottom central flange 38(10). The bottom central flange 38(10) is enlarged in surface area as compared to previous embodiments, and subsumes the role of left bottom corner flange 36 and right bottom corner flange 37 in other embodiments. As with the immediately preceding embodiment, first end of chamber housing 132 serves as outlet tube 84(10) by extending above the bottom central flange 38. A second end of chamber housing 132, which extends upwardly from the pump body 50(10), sealing punctures or otherwise protrudes in sealed fashion through an aperture 170 provided or formed on front layer 24 (see FIG. 10B). In order to allow movement of both diaphragms of the pump motive assembly 50(10), the pump motive assembly 50(10) can be secured or mounted to bottom central flange 38(10) via outlet tube 84(10), e.g., a bonding or adhesion of outlet tube 84(10) to bottom central flange 38(10).

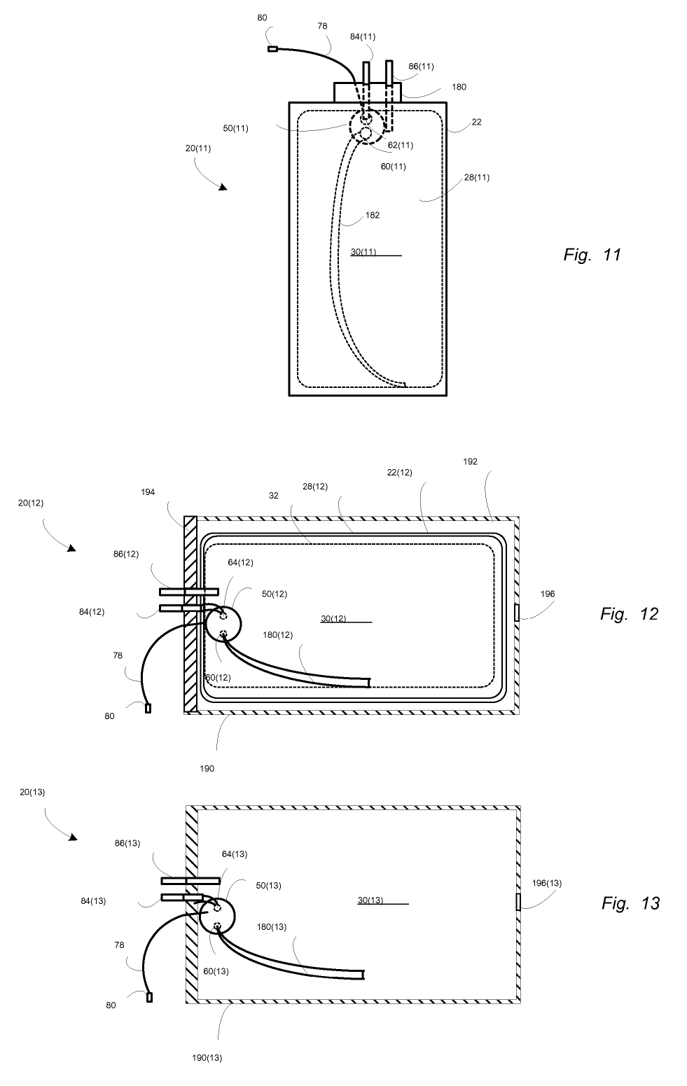

The example embodiments already described basically depict the integrated pump motive assembly as being situated or positioned in a lower (e.g., with respect to gravity) location on or in the container body. Such need not be the case, as the pump motive assembly can be located essentially anywhere within the fluid reservoir or elsewhere on the container body. For example, the illustrative embodiment of FIG. 11 shows a pump motive assembly 50(11) which is suspended, mounted, or otherwise positioned at or near the top of fluid reservoir 30(11). In disposable fluid container 20(11), the electrical lead 78, outlet tube 84(11), and fill tube 86(11) all extend through top central flange 180, in similar manner as did comparable interfaces through bottom central flange 38 in previous embodiments (e.g., sandwiched between front layer 24 and rear layer 26). The outlet tube 84(11) extends upwardly from outlet port 62(11) of pump motive assembly 50(11). In order to feed fluid from near the bottom of an emptying container body, a feed tube 182 is connected to inlet port 60(11) of pump motive assembly 50(11), with a distal end of feed tube 182 preferably reaching or extending to or near a bottom of fluid reservoir 30(11).

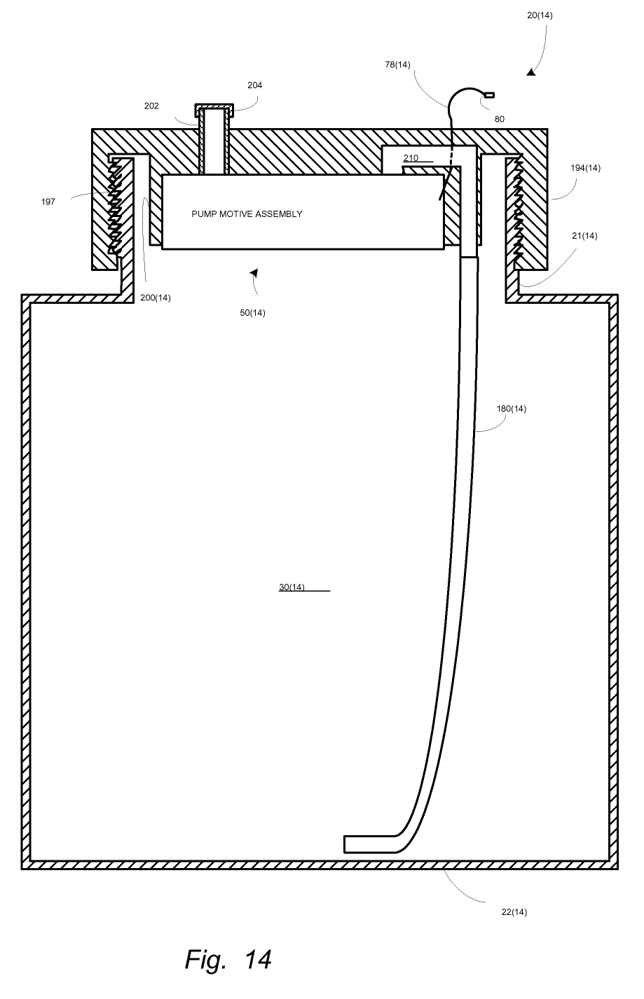

The example disposable fluid container 20(12) of FIG. 12 shows a container body 22(12) having an orientation other than vertical. The horizontally oriented disposable fluid container 20(12) of FIG. 12 further illustrates that, in other example embodiments, disposable fluid container can be inserted into or even comprise a container housing 190 for at least partially enclosing the collapsible bladder. In the illustrated example, container housing 190 comprises a housing main body 192 and a housing top or lid 194. The housing top or lid 194 has (sealed) apertures through which outlet tube 84(12), fill tube 86(12), and electrical lead 78 can all extend. In order to feed fluid from near the bottom of an emptying container body, a feed tube 182(12) is connected to inlet port 60(12) of pump motive assembly 50(12) in like manner as the previously described embodiment, e.g., with a distal end of feed tube 182(12) preferably reaching or extending to or near a bottom of fluid reservoir 30(12). In some variations and applications of this embodiment, the container housing 190 can be reusable, while in other variations and applications the container housing 190 can be integral with or at least supplied and disposed with the disposable fluid container 20(12) itself. In some implementations of this embodiment, the container housing 190 is substantially rigid. When rigid, the container housing 190 has a bleed valve 196 or other comparable opening to permit collapsing of the bladder.

In the example disposable fluid container 20(13) of FIG. 13, container housing 190(13) is one and the same with container body 22(13), there being no internal pouch or bag to define fluid reservoir 30(13). Rather, container housing 190(13) defines fluid reservoir 30(13). The container housing 190(13) can be a two piece housing having a cap or lid and bottom, in like manner as container housing 190(12). The pump motive assembly 50(13) is suspended to or mounted on a lid or active end of container housing 190(13). The container housing 190 is preferably rigid, and as such as bleed valve 96 or other comparable opening to permit discharge of fluid as withdrawn via pump motive assembly 50(13).

In the example disposable fluid container 20(14) of FIG. 14, container 22(14), includes a container body 22(14) and a lid 194(14). Although not illustrated as such in FIG. 14, in differing implementations the container body 22(14) can either have a collapsible internal pouch or bag to define a fluid reservoir 30(14), or be rigid construction (and likely have a bleed valve or the like).

In the embodiment of FIG. 14, a generic pump motive assembly 50(14) is attached to or integrated in lid 194(13). It so happens that the container body 22(14) of FIG. 14 is of a type that has a narrowed, threaded neck 21(14) for defining a mouth which is covered by lid 194(14). In particular, the mouth of container body 22(14) has lid 194(14) screwed thereon by virtue of counterthreads 197 or the like formed on the interior periphery of lid 194(14). However, it should be understood that the configuration of the container body mouth, or the manner of engagement of the container mouth by lid 194(14) is not limiting, but that other configurations and engagement techniques can be utilized, such as press-fit of the lid, sealing or adhering of the lid to the container mouth, or fastening of the lid to the container mouth, as a few examples.

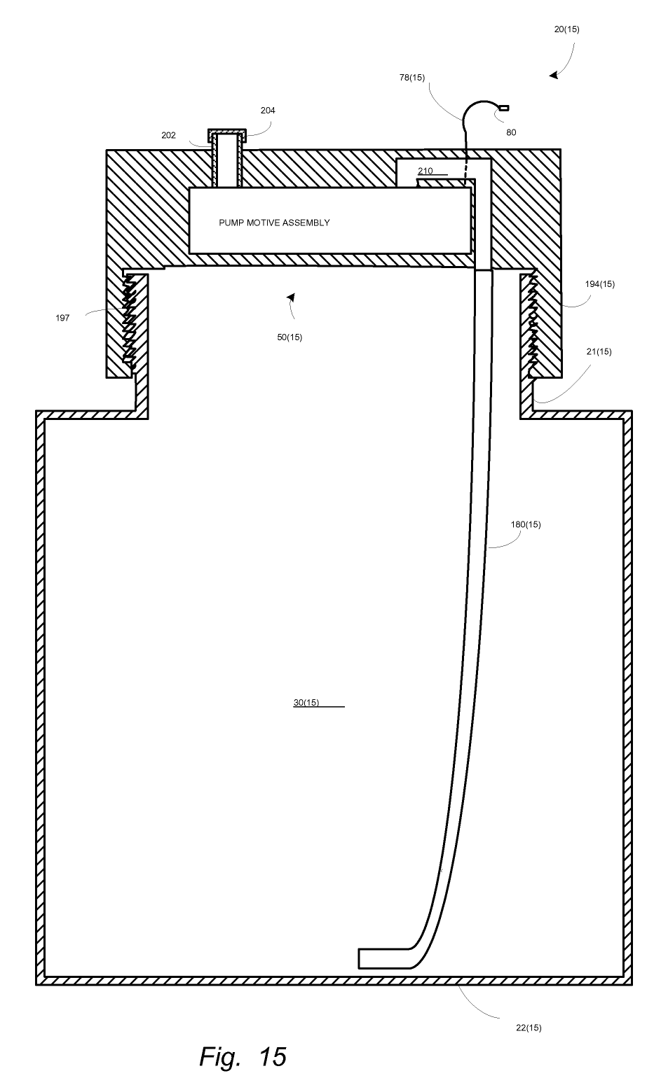

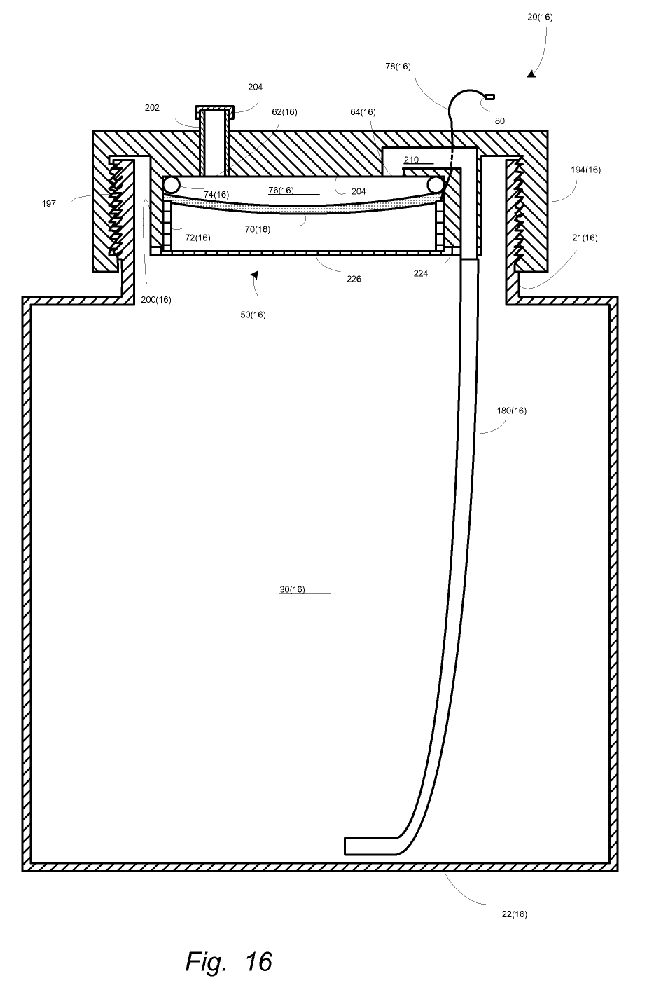

The lid 194(14) has a pump region 200(14) which depends internally into the neck of the container body 22(14) from an underside of the lid 194(13). However, in other implementations the pump region can be situated above the neck of the container body, as illustrated in FIG. 15 by lid 194(15) with its pump region 200(15). In the example illustration, pump region 200(14) has an essentially disk or cylindrical shape, but other shapes are possible.

The lid 194(14) has an outlet port 202 through which fluid is selectively discharged by action of generic pump motive assembly 50(14) after being drawn from reservoir 30(14). In the example implementation of FIG. 14, the outlet port 202 takes the form of a tube or duct which extends through a thickness of lid 194(14). A first end of the tube or duct forming outlet port 202 is connected to an outlet port of the pump motive assembly 50(14). A second end of the tube or duct forming outlet port 202, in the illustrated example, protrudes slightly beyond an upper surface of lid 194(14). Preferably, when the pump motive assembly 50(14) is not in use, the second end of outlet port 202 is covered or shut. Such covering or shutting of the second end of outlet port 202 is represented generically in FIG. 14 by a seal or cap 204. It should be realized that seal or cap 204 can take various implementations and be either removable, pierceable, or opened for permitting egress of fluid from outlet port 202. Further, in other implementations, the outlet port 202 may have other features, location, or degree of protrusion (or no protrusion) relative to lid 194(14).

The lid 194(14) also has an inlet channel 210 through which fluid is communicated from fluid reservoir 30(14) to an input port of pump motive assembly 50(14). In the particular implementation shown in FIG. 14, the inlet channel 210 is internally provided in lid 194(14). Preferably the inlet channel 210 is connected to a tube or hose 180(14) which extends below lid 194(14) into reservoir 30(14) for drawing fluid from the reservoir into the pump motive assembly 50(14).

In addition to outlet port 202 and inlet channel 210, lid 194(14) has an electrical lead 78(14) extending therethrough for providing an electrical signal to pump motive assembly 50(14). The electrical lead 78(14), which can have an electrical terminal 80 thereon in same manner as other embodiments, may protrude beyond the top surface of lid 194(14) as shown, or terminate in a terminal flush with the top surface or integral with lid 194(14), or have other configurations.

FIG. 16 illustrates that the pump motive assembly in the lid can comprise a piezoelectric diaphragm, such as piezoelectric diaphragm 70(16). As previously explained for other embodiments, a piezoelectric diaphragm is just one example of a suitable displaceable electrodynamic actuator for use with the pump motive assembly. In the example of FIG. 16, the lid 194(16) partially defines a pump housing. Particularly does an underside surface of lid 194(16) in the pump region 200(16) provide a pump housing chamber wall 220 upon which a pump outlet and pump inlet communicate with pumping chamber 76(16). The pump housing is further defined by sidewall 224. Pump housing sidewall 224 may be cylindrical in shape, or any other shape which can be accommodated within the interior of lid 194(16). The pump housing also includes a housing cover wall 226 which fits over the pump and separates the pump from contents of the fluid reservoir 30(16).

The piezoelectric diaphragm member 70(16) is circumferentially retained in the pump housing between coverall wall 226 and the chamber wall 220, e.g., by spacer 72(16) and O-ring or spacer 74(16). On its underside piezoelectric diaphragm member 70(16) defines the pumping chamber 76(16), which exists between piezoelectric diaphragm member 70(16) and chamber wall 220. An outlet port 62(16) and inlet port 64(16) of the pump housing are oriented so that inlet and outlet fluid flows are essentially axial relative to the piezoelectric diaphragm 70(16). Unillustrated valves may be provided for one or both of outlet port 62(16) and inlet port 64(16). As previously explained, the piezoelectric diaphragm member 70(16) can be a ruggedized laminated piezoelectric member comprising a piezoelectric wafer core.

It should be understood that the pump motive assembly 50(16) with its piezoelectric diaphragm member 70(16) may be provided within the neck of the container 20(16) in the manner shown in FIG. 16, or above the neck of the container in a manner comparable to that illustrated in FIG. 15.

In operation, fluid from reservoir 30(16) is drawn through tube or hose 180(14) and through inlet channel 210 into pumping chamber 76(16) by action of piezoelectric diaphragm member 70(16), as piezoelectric diaphragm member 70(16) is operated in accordance with the electrical signal applied on electrical lead 78(14). Movement or defection of piezoelectric diaphragm member 70(16) away from chamber wall 220 serves to draw fluid into pumping chamber 76(16) through inlet 64(16). On the other hand, movement or deflection of piezoelectric diaphragm member 70(16) away from chamber wall 220 serves to exhaust or expel fluid from pumping chamber 76(16) through outlet port 62(16). The amount of discharge of fluid from container 22(16) thus can be closely regulated by controlled operation of piezoelectric diaphragm member 70(16).

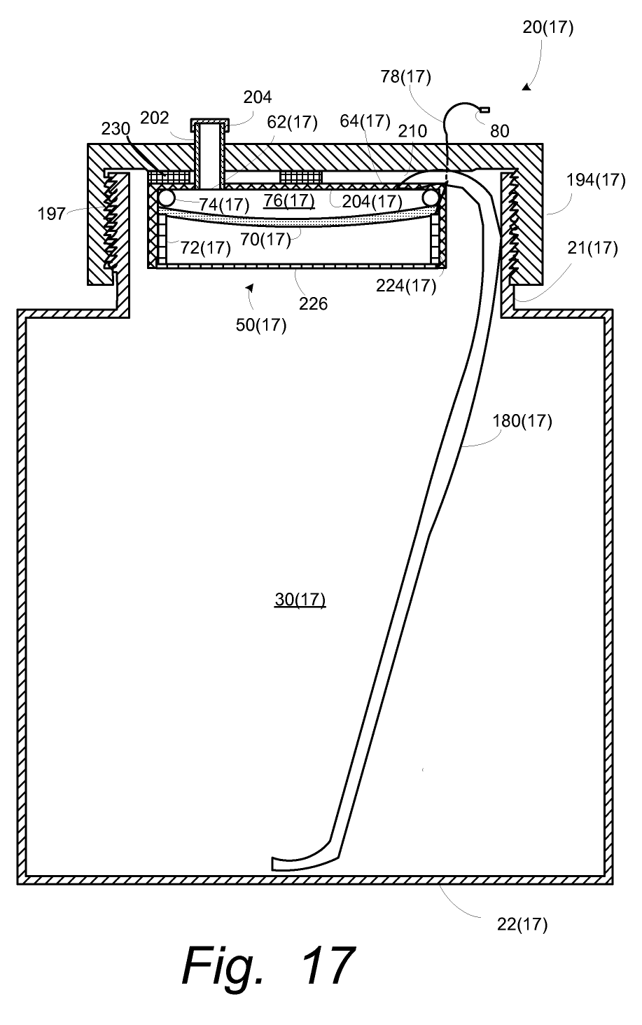

The example disposable fluid container 20(17) of FIG. 17 resembles the container of FIG. 16, with one notable exception being that the pump motive assembly 50(17) is attached to the underside surface of lid 194(17) rather than being at least partially enclosed by the lid. The pump motive assembly 50(17) can be slightly spaced away from the underside surface of lid 194(17) in the manner shown in FIG. 17, or lie flush against the underside surface of lid 194(17). In the FIG. 17 implementation, one or more standoffs 230 may be provided for attaching the pump motive assembly 50(17) to the underside surface of lid 194(17). The standoffs may be fasteners. In the case in which the pump motive assembly 50(17) lies flush against the underside surface of lid 194(17), an adhesive or epoxy may be utilized to facilitate attachment.

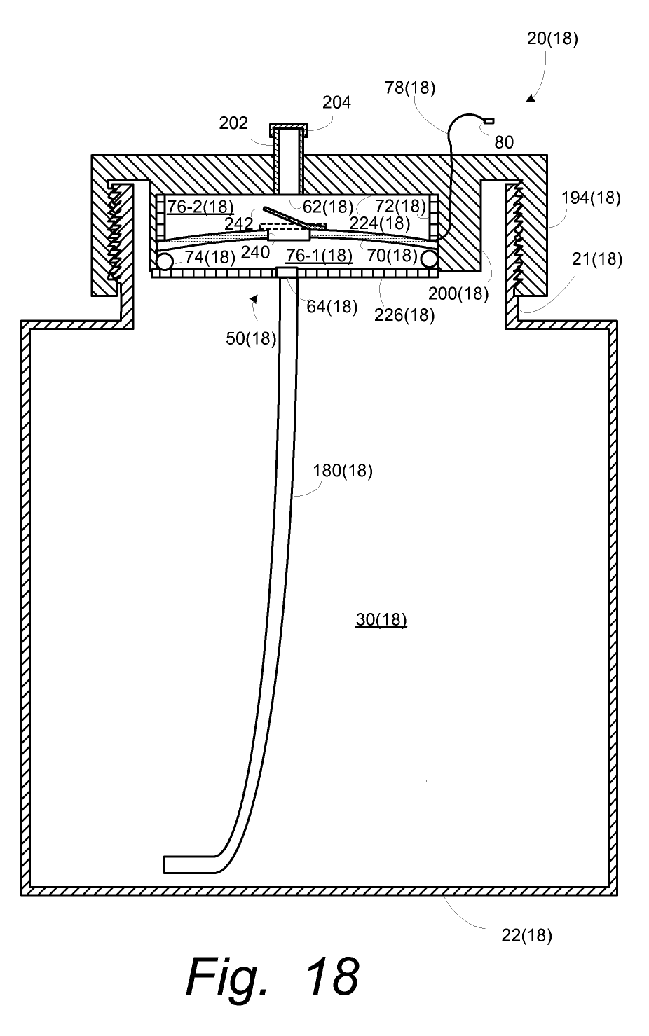

FIG. 18 illustrates yet another embodiment of a disposable fluid container, particularly container 20(18). Among other things, the pump motive assembly 50(18) provided in the lid 194(18) of container 20(18) differs from that of earlier described embodiments in that the piezoelectric diaphragm member 70(18) has a port 240 which is selectively covered by a valve 242. The pump motive assembly 50(18) is situated in a pump region 200(18) of lid 194(18). The pump region 200(18) has a recess formed therein, preferably disk-shaped, with pump housing chamber wall 220 being formed by a underside surface of lid 194(18) in the recess. The pump housing also includes a housing cover wall 226(18) which fits over the pump and separates the pump from contents of the fluid reservoir 30(18). The piezoelectric diaphragm member 70(18) is circumferentially retained in the pump housing between coverall wall 226(18) and the chamber wall 220(18), e.g., by spacer 72(18) and O-ring or spacer 74(18). In the FIG. 18 embodiment, separate fluid chambers 76-1(18) and 76-2(18) are formed on inlet sides and outlet sides, respectively, of the piezoelectric diaphragm member 70(18). An outlet port 62(18) is formed in lid 194(18); inlet port 64(18) of the pump housing is formed on coverall wall 226(18). The inlet port 64(18), the outlet port 62(18), and the aperture port 240 are preferably axially aligned. The pump outlet port 62(18) is aligned with lid outlet port 202. Fluid can be drawn into fluid chambers 76-1(18) through tube or hose 180(18) by the action of piezoelectric diaphragm member 70(18), and then upon opening of valve 242 flow through port 240 into fluid chamber 76-2(18) and discharged through outlet port 62(18) and lid outlet port 202.

It should be understood that the lid outlet port 202 can either project from the lid 194(18) as illustrated, or be flush with a top surface of the lid. Other configurations of lid outlet port structure are also possible, as well as means for sealing the same.

The action of piezoelectric diaphragm member 70(18) with its selectively covered (by valve 242) port 240 is understood with reference to U.S. patent application Ser. No. 11/279,645, filed Apr. 13, 2006, entitled “Piezoelectric Actuator and Pump Using Same”, which is incorporated herein by reference in its entirety.

Although not specifically shown and discussed as such, it should be understood that all embodiments and implementations described herein can optionally include an identification device for storing container information in electronic form, in like or similar manner to that previously described. Moreover, each embodiment can be provided with a pump motive assembly situated in a lid portion which is either above a neck of the container or in the neck of the container. Further, each pump motive assembly can either be internally situated within the lid, or attached to the lid.

Some of the preceding embodiments have been described from a perspective that the disposable fluid containers are formed by the bonding of multi-ply or multi-layers, typically after the pump motive assembly (in some embodiments including, e.g., outlet tube 84, fill tube 86, and electrical lead 78) have been positioned between films such as front layer 24 and rear layer 26, for example. Such bonding can be by application of electromagnetic energy or heat, being careful not to deform or damage the pump motive assembly and the other components. Yet layered bonding is not the exclusive mode of manufacture, since in other modes a collapsible bladder having but one open end can be preformed to have the pump motive assembly inserted therein. In such insertion mode, sealed apertures need to be provided so that components such as outlet tube 84, fill tube 86, and electrical lead 78 can extend from inside the collapsible bladder 28 to the exterior. Appropriate sealing structure and techniques are well within the ken of the person skilled in the art. In yet other modes, an injection molding process can also incorporate the pump motive assembly as an integral part of the disposable fluid container.

In some embodiments the container body 22 (defining bladder 28 in some embodiments) is formed from flexible material. Any suitable flexible material can be utilized which collapses as fluid is withdrawn therefrom. The choice of material may depend upon field of application (with possible attendant concern for how the material interfaces with the stored fluid) as well as possible environmental concerns. Example materials include, but are not limited to, plasticized polyvinylchloride (PVC), ethylene vinylacetate, polypropylene, and copolyester ether, for example.

The disposable fluid containers described in the illustrated embodiments and other embodiments encompassed hereby can be utilized in many applications and fields of endeavor. Non-limiting and non-exhaustive examples include disposable medical applications (intravenous bag, blood bag, TPN (Total Parenteral Nutrition) bags, insulin containers, medicament bag, sterile dosing applications, infusion devices), disposable consumer applications; disposable food service items (e.g., beverage) for, e.g., guaranteed compatibility or inventory control; industrial or agricultural (e.g. pesticide, insecticide, or fertilizer) delivery or dispensing of fluids.

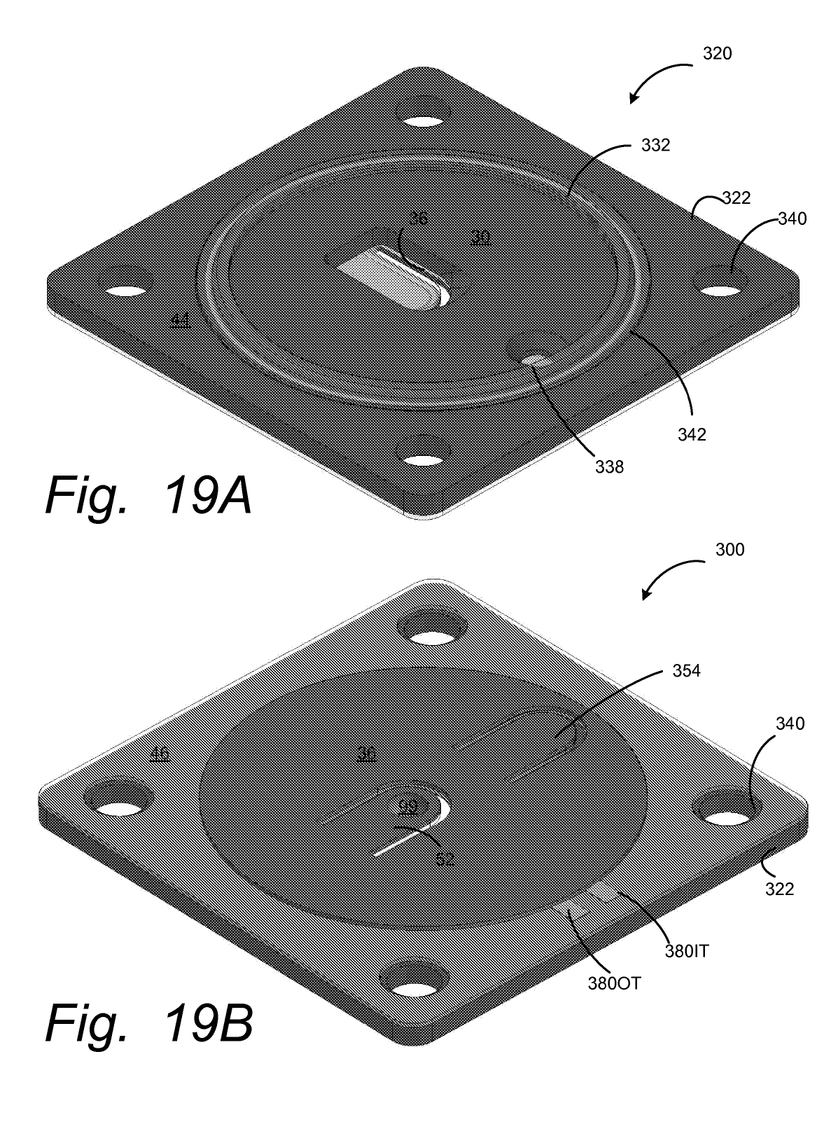

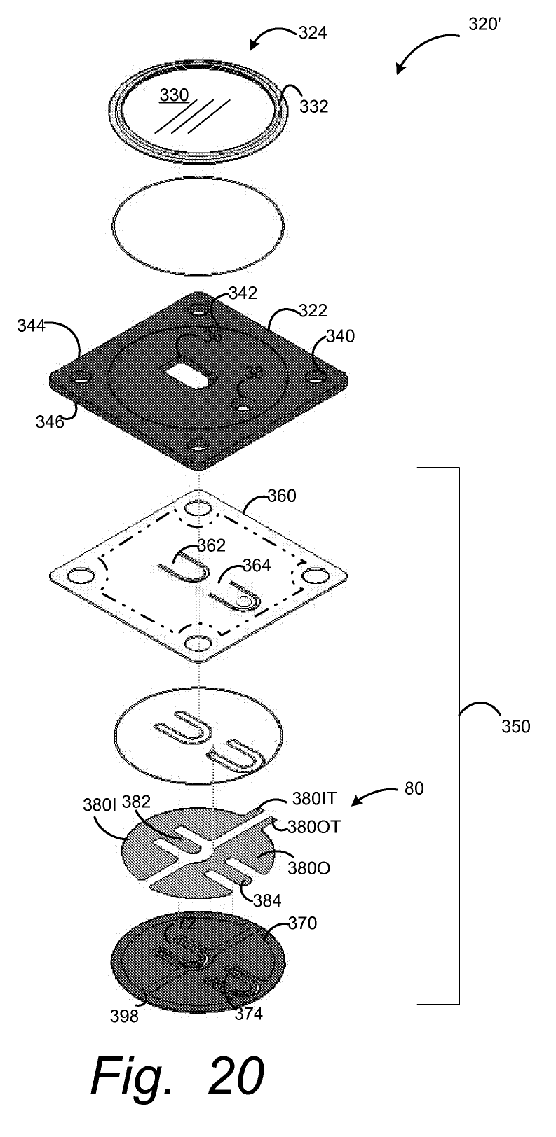

FIG. 19 and FIG. 20 illustrate another embodiment of a piezoelectric pump motive assembly 300 which can be utilized, in any of the foregoing embodiments as well as other embodiments encompassed hereby, in lieu of the pump motive assemblies devices actually illustrated. FIG. 19 and FIG. 20 particularly show a pump 320 which is comprised of a base plate or pump base member 322 and a diaphragm layer 324. The diaphragm layer 324 covers at least a portion of the base member and defines a pumping chamber 326 between the pump base member 322 and the diaphragm layer 324. The diaphragm layer 324 comprises a piezoelectric central region 330 selectively deformable upon application of an electrical signal for pumping fluid into and out of the pumping chamber. A peripheral region 332 (which, in at least some embodiments, can be an electromagnetically transmissive region) essentially surrounds the central piezoelectric region 30. The diaphragm layer 324 is bonded or otherwise secured to the pump base member 322 (to cover pumping chamber 326). For example, electromagnetic bonding may be utilized in accordance with the teachings of U.S. patent application Ser. No. 11/104,662 filed Apr. 13, 2005, by Clayton Ball, entitled “ELECTROMAGNETICALLY BONDED PUMPS AND PUMP SUBASSEMBLIES AND METHODS OF FABRICATION”, which is incorporated by reference herein in its entirety.

Pump base member 322 has an inlet port 336 and an outlet port 338 formed therein. The inlet port 336 and outlet port 338 are formed entirely through the thickness of pump base member 322, so that fluid can enter through inlet port 336 into pumping chamber 326 and so that fluid can exit pumping chamber 326 through outlet port 338. In the particular example illustrated in FIG. 19A, FIG. 19B, and FIG. 20, inlet port 336 has an essentially U shape, while outlet port 338 has a circular or slightly elliptical shape. Differing shapes for inlet port 336 and outlet port 338 are certainly possible in other implementations. The number of ports provided in pump base member 322 is not confined to two, since a greater of ports may instead be provided such as, for example, two or more inlet ports and/or two or more outlet ports.

By way of non-limiting example, the pump base member 322 of the embodiment of FIG. 19A, FIG. 19B, and FIG. 20 takes the form of an essentially flat (planar) plate having a substantially square shape. Proximate each of its four corners, pump base member 322 has through holes or fastening apertures 340. The fastening apertures 340 may be used to secure an unillustrated and optional pump cover or housing to pump base member 322 for protecting and encasing diaphragm layer 324. Other ways for connecting a pump cover or housing (if desired) are also possible.

In addition, pump 320 comprises a valve subassembly 350 which is also shown in exploded fashion in FIG. 20. Valve subassembly 350 can be electromagnetically welded to valve interface surface 346 of the pump base member 322 for providing an inlet valve 352 which selectively opens and closes the inlet port 336 and an outlet valve 354 which selectively opens and closes the outlet port 338. Valve subassembly 350 comprises a pump interface layer 360 having an interface layer inlet flap 362 and an interface layer outlet flap 364; a subassembly cover layer 370 having a cover layer inlet flap 372 and a cover layer outlet flap 374; and, an intermediate layer 380 positioned between interface layer 360 and cover layer 370, intermediate layer 380 having an intermediate layer inlet flap 382 and an intermediate layer outlet flap 384.

Intermediate layer 380 may comprise two discrete and separated segments, e.g., intermediate layer segment 3801 and intermediate layer segment 3800. The intermediate layer segment 3801 bears intermediate layer inlet flap 382, the intermediate layer segment 3800 bears intermediate layer outlet flap 384. A tab 3801T is provided at a circumference portion of intermediate layer segment 3801, and similarly a tab 3800T is provided at a circumference portion of intermediate layer segment 3800. The tabs 3801T and 3800T may be used as electrical leads in an implementation in which the segments of the intermediate layer 380 are metallic and are connected to receive an electrical signal.

Another pump motive assembly is described in context of an integrated thermal treatment system in U.S. patent application Ser. No. 11/104,668 filed Apr. 13, 2005, by Clayton Ball, entitled “INTEGRATED THERMAL EXCHANGE SYSTEMS AND METHODS OF FABRICATING SAME”, which is incorporated herein by reference in its entirety.

As mentioned above, the pump motive assembly utilized in any of the foregoing embodiments or other embodiments encompassed herein can use any type of displaceable electrodynamic actuator. Although the foregoing example embodiments primarily depict displaceable electrodynamic actuators which comprise a piezoelectric diaphragm, other types of displaceable electrodynamic actuators can be utilized in lieu thereof. These other types of displaceable electrodynamic actuators are known and therefore the person skilled in the art will understand how to implement other types of displaceable electrodynamic actuators. For example, actuators formed using electroreactive polymer(s) (EAP), electrorestrictive members, actuators comprised of memory alloys, magneto-restrictive elements are understood from one or more of the following (all of which are incorporated herein by reference in their entirety): US Patent Publication 2004/0234401 of Banister; US Patent Publication 2003/0072982 of Takeuchi et al.; US Patent Publication 2004/0209133 to Hirsch; US Patent Publication 2004/0199104 to Ujhelyi et al.; US Patent Publication 2004/0163622 to Sakaguchi; and US Patent Publication 2004/0236171 to Rastegar et al. In essence, displaceable electrodynamic actuator encompasses any “smart” material which can used applied electrical energy to yield a mechanical displacement or deformation of itself, and (preferably) when subject to a mechanical force produces an electrical current.

Further, although for sake of simplicity the container bodies of the disposable fluid containers described herein have been described and illustrated as comprising only two plys of layers of film, it should be understood that a greater number of layers or plys can be utilized, and that the layers or plys may differ in composition and character.

The pump motive assemblies described herein advantageously can be made small enough and inexpensively so that they can comprise the disposable fluid container. The pump motive assemblies can be utilized as integral part of the disposable fluid container. Prior art devices could not be so integrated because of factors such as size, complexity, and the need for additional electrical or mechanical actuation.

Although various embodiments have been shown and described in detail, the claims are not limited to any particular embodiment or example. None of the above description should be read as implying that any particular element, step, range, or function is essential such that it must be included in the claims scope. The scope of patented subject matter is defined only by the claims. The extent of legal protection is defined by the words recited in the allowed claims and their equivalents. It is to be understood that the invention is not to be limited to the disclosed embodiment, but on the contrary, is intended to cover various modifications and equivalent arrangements.

What is claimed is:

1. A disposable fluid container comprising:
a container for containing a fluid;
a fluid-contacting pump motive assembly which is integrated with the container for pumping fluid from an interior of the container.

2. The apparatus of claim 1, wherein the container comprises a container body, and wherein the fluid-contacting pump motive assembly is integrated with the container body for pumping fluid from the interior of the container.

3. The apparatus of claim 1, wherein the container comprises a container body and a container lid, and wherein the fluid-contacting pump motive assembly is integrated with the container lid for pumping fluid from the interior of the container.

4. The apparatus of claim 1, wherein the pump motive assembly is connected to receive pump driving signals from outside the container.

5. The apparatus of claim 1, further comprising:
an identification device for storing container information in electronic form;
an electrical terminal or connector through which the container information stored in the identification device is accessible.

6. The apparatus of claim 5, wherein the container information is one of container identification information, container fluid contents information, container volume information, lot number, serial number, origination (born on) date, and expiration date.

7. The apparatus of claim 5, wherein the pump motive assembly comprises a housing, and wherein the identification device is mounted to or within the pump housing.

8. The apparatus of claim 1, wherein the pump motive assembly comprises a displaceable electrodynamic actuator.

9. The apparatus of claim 8, wherein the pump motive assembly comprises:
a pump housing;
a displaceable electrodynamic actuator situated internally in the pump housing.

10. The apparatus of claim 8, wherein the pump motive assembly comprises plural piezoelectric diaphragms which are edge bonded to form a bellows chamber.

11. The apparatus of claim 1, wherein the container body comprises a collapsible bladder, and wherein the pump motive assembly is situated at least partially exteriorly of the bladder.

12. The apparatus of claim 1, wherein the container body comprises a collapsible bladder, and wherein the pump motive assembly is situated at least partially within the bladder.

13. The apparatus of claim 1, wherein the container comprises a container body, and wherein the container body comprises plural layers with the pump motive assembly being situated between two of the plural layers.

14. The apparatus of claim 13, wherein the pump motive assembly is situated in a collapsible bladder defined by the two of the plural layers.

15. The apparatus of claim 13, wherein the pump motive assembly is embedded in the container housing by bonding of the two of the plural layers.

16. The apparatus of claim 1, wherein the container body comprises a lid, and wherein the pump motive assembly is connected or attached to the lid.

17. A disposable fluid container comprising:
a container body for containing a fluid;
a fluid-contacting pump motive assembly which is substantially internal to the container body for pumping fluid from an interior of the container body.

18. The apparatus of claim 17, wherein the pump motive assembly is connected to receive pump driving signals from outside the container.

19. The apparatus of claim 17, further comprising:
an identification device for storing container information in electronic form;
an electrical terminal through which the container information stored in the identification device is accessible to the host device.

20. The apparatus of claim 19, wherein the container information is one of container identification information, container fluid contents information, container volume information, lot number, serial number, origination (born on) date, and expiration date.

21. The apparatus of claim 17, wherein the pump motive assembly comprises a displaceable electrodynamic actuator.

22. The apparatus of claim 21, wherein the pump motive assembly comprises:
a pump housing;
a displaceable electrodynamic actuator situated internally in the pump housing.

23. The apparatus of claim 21, wherein the pump motive assembly comprises plural piezoelectric diaphragms which are edge bonded to form a bellows chamber.

24. The apparatus of claim 17, wherein the container body comprises a collapsible bladder, and wherein the pump motive assembly is situated at least partially exteriorly of the bladder.

25. The apparatus of claim 17, wherein the container body comprises a collapsible bladder, and wherein the pump motive assembly is situated at least partially within the bladder.

26. The apparatus of claim 17, the container body comprises plural layers with the pump motive assembly being situated between two of the plural layers.

27. The apparatus of claim 26, wherein the pump motive assembly is situated in a collapsible bladder defined by the two of the plural layers.

28. The apparatus of claim 26, wherein the pump motive assembly is embedded in the container housing by bonding of the two of the plural layers.

29. A disposable fluid container comprising:
a container for containing a fluid, the container comprising a container body and a container lid;
a fluid-contacting pump motive assembly which is integrated within or attached to the container lid for pumping fluid from an interior of the container body.

30. The apparatus of claim 29, wherein the pump motive assembly is connected to receive pump driving signals from outside the container.

31. The apparatus of claim 29, further comprising:
an identification device for storing container information in electronic form;
an electrical terminal or connector through which the container information stored in the identification device is accessible.

32. The apparatus of claim 31, wherein the container information is one of container identification information, container fluid contents information, container volume information, lot number, serial number, origination (born on) date, and expiration date.

33. The apparatus of claim 29, wherein the pump motive assembly comprises a displaceable electrodynamic actuator.

34. The apparatus of claim 33, wherein the pump motive assembly comprises:
a pump housing, a portion of the pump housing being formed by the container lid;
a displaceable electrodynamic actuator situated internally in the pump housing.
